(19) United States
(12) Patent Application Publication
(10) Pub. No.: US 2003/0212248 A1
(43) Pub. Date: Nov. 13, 2003

(54) Process to increase protein stability

(76) Inventor: Thomas Charles Furman, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/311,782

(22) PCT Filed: Jun. 29, 2001
(86) PCT No.: PCT/US01/16510

(30) Foreign Application Priority Data
Jul. 12, 2000 (US) 60217578

(51) Int. Cl. C07K 14/615

(52) U.S. Cl. 530/303

(57) ABSTRACT

This invention relates to a process for increasing the stability of a protein solution in which a folded protein, a contaminating protein, which possesses a free thiol moiety, and a low molecular weight thiol compound are present. The process entails adding an effective amount or, preferably, an optimal level of an oxidizing agent to the protein solution.

This invention is in the field of processes for producing proteins that are useful for treating various diseases.

Proteins are used as pharmaceuticals for the treatment of diseases in humans and other animals. Many pharmaceutical proteins contain cysteine residues. In proteins comprising more than one cysteine residue, the thiol groups of the cysteine residues are usually covalently linked with other cysteine residues by specifically paired disulfide bonds.

Proteins may be made by recombinant DNA technology in host cells in which correct disulfide bond formation does not take place. For these proteins, a folding reaction is required to form the proper disulfide bonds.

Many procedures for maximizing the yield of correctly formed disulfide bonds in protein folding reactions have been disclosed. For example, Niwa, et al., U.S. Pat. No. 5,102,985, issued Apr. 7, 1992, treated solutions of small proteins in reduced form with hydrogen peroxide to generate proteins having an intramolecular disulfide bridge in 90-96% yield.

Despite numerous advances in producing correctly paired disulfide bonds in proteins in high yield, folding reactions usually do not result in quantitative yield of the properly disulfide-paired protein. As a result, side products such as contaminating proteins, which have one or more of their cysteine residues in unpaired, free thiol form, are generated along with the correctly folded target protein.

Such contaminating proteins may also be derived from a properly folded protein. Volkin, D. B., et al., in a review in Molecular Biotechnology, 8:105-122 (1997), reported that correctly paired disulfide bonded cysteine residues themselves are quite labile under certain conditions, such as alkaline pH. With model peptides such as cystine and glutathione, it was noted that the resulting free thiol groups catalyze disulfide interchange reactions. These reactions are accelerated by exogenous thiols and are inhibited by reagents that block thiol groups.

In order to generate highly purified therapeutic proteins useful for treating various diseases in humans and other animals, a protein must undergo numerous processing steps after folding. These steps may include filtration, concentration and purification procedures, enzymatic and chemical cleavage reactions, and storage in solution.

I have found that during such protein processing steps, the free thiol groups in a contaminating protein can generate mispaired intramolecular and intermolecular disulfide bonds and initiate disulfide interchange reactions that scramble previously correctly-formed disulfide bonds. These reactions lower the yield of the purified target protein and limit the conditions under which storage, purification, concentration and other processing steps can be conducted. They may also lead to the need for extra purification steps to remove newly-formed contaminating protein.

I have discovered a process that solves the problems noted above. This process increases the stability of a folded protein in solution and provides a higher yield of the protein during post-folding storage, concentration and other processing steps. As a result, greater quantities of highly purified proteins, including therapeutic proteins useful in treating various diseases in humans and other animals, are obtained.

Accordingly, the instant invention provides a process for increasing the stability of a folded protein in a solution further comprising a contaminating protein possessing one or more free thiol groups. The process involves adding a low molecular weight thiol compound and an effective amount of an oxidizing agent to the protein solution to form mixed disulfide adducts between the thiol groups of the contaminating protein and the low molecular weight thiol compound.

The present invention also provides a process for increasing the stability of a folded protein in a solution further comprising a contaminating protein, possessing one or more free thiol groups, and also comprising a low molecular weight thiol compound. The process involves adding an effective amount of an oxidizing agent to the protein solution to form mixed disulfide adducts between the thiol groups of the contaminating protein and the low molecular weight thiol compound.

The present invention also provides a process for making human insulin or an insulin analog. The process involves adding a low molecular weight thiol compound and an effective amount of an oxidizing agent to a protein solution comprising a folded precursor of human insulin or a folded precursor of an insulin analog, and a contaminating protein, and then converting the folded precursor protein to human insulin or an insulin analog.

The present invention also provides another process for making human insulin or an insulin analog. This process involves adding an effective amount of an oxidizing agent to a protein solution comprising a folded precursor of human insulin or a folded precursor of an insulin analog, a contaminating protein and a low molecular weight thiol compound, and then converting the folded precursor protein to human insulin or an insulin analog.

Upon storage over time, protein solutions stabilized by the process of the present invention retain a greater quantity of the correctly folded protein than untreated solutions. Furthermore, protein solutions stabilized by the process of this invention generate a higher yield of the target protein during subsequent processing steps such as filtration, concentration, purification, and enzymatic and chemical cleavage. Thus, the invention provides a greater quantity of properly folded proteins, including therapeutic proteins useful for treating diseases in humans and other animals.

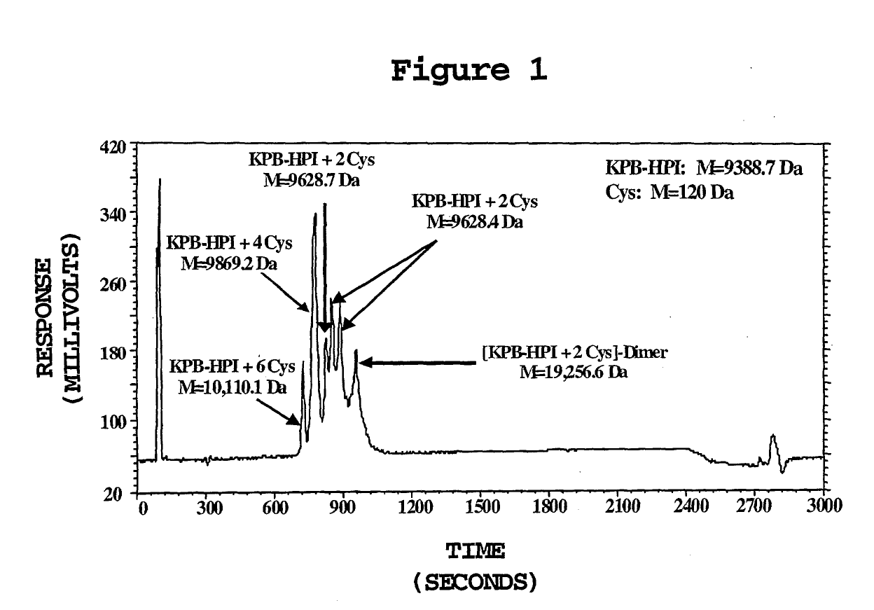

FIG. 1 shows mass spectrometry analysis of the protein peak of an HPLC chromatographed reaction solution comprising a folded protein (KPB-HPI), cysteine and hydrogen peroxide indicating the presence of mixed disulfide adducts.

As used herein, the word “stability” refers to the relative resistance of a protein in solution to chemically or physically degrade over time. An increase in stability means the soluble concentration of a folded protein decreases more slowly over time or decreases to a lesser extent during a specified procedure compared to a control solution.

The word “protein” refers to a compound composed of strands of 10 or more amino acids connected by peptide bonds in which at least one of the amino acid residues is cysteine. Preferably, the protein is composed of strands of 20 or more amino acid residues connected by peptide bonds in which at least one of the amino acid residues is cysteine. A protein may contain one or more strands of amino acids connected together by covalent bonds, such as disulfide bonds, or by non-covalent interactions. Proteins of the present invention include precursor proteins and analogs thereof, which may be converted into useful therapeutic proteins or analogs thereof, by procedures including, inter alia, chemical and enzymatic cleavage reactions.

The term “therapeutic protein” refers to a protein that has a demonstrated biological activity and may be delivered to a patient in need thereof by an acceptable route of administration. The biological activity of a therapeutic protein results from interaction of the protein with receptors and/or other intracellular or extracellular targets leading to a biological effect and may be demonstrated using in vitro or in vivo techniques.

Examples of therapeutic proteins include hormones, antibodies and enzymes. See, for example, Platz, R. M. et al., in U.S. Pat. No. 6,051,256, issued Apr. 18, 2000, which includes in Table 1 a list of many therapeutic proteins and their indications.

As used herein, protein hormones include, inter alia, colony stimulating factors, such as granulocyte colony stimulating factor and macrophage colony stimulating factor; poietins such as erythropoetin (EPO) and thrombopoietin; growth factors, such as growth hormone releasing factor, epidermal growth factor, fibroblast growth factor, hepatocyte growth factor, insulin-like growth factors and nerve growth factor; growth hormones, such as human growth hormone; interferons, such as interferon-alpha-2a, interferon-alpha-2b, interferon-beta-la, interferon-beta-1b, interferon-alpha-n3 and gamma-interferon; interleukins, such as interleukin-1, interleukin-3, interleukin-4, interleukin-6, interleukin-10, interleukin-11 and interleukin-12; metabolic hormones such as proinsulin, insulin, leptin and amylin; fertility hormones, such as follicle stimulating hormone (FSH), leutinizing hormone and chorionic gonadotropin; stem cell factors; and miscellaneous hormones such as ciliary neurotrophic factor, alpha-1-antitrypsin and calcitonin.

As used herein, the word “antibodies” refers to glycoproteins which bind to antigens. Therapeutic antibodies include, inter alia, monoclonal antibodies, IgA, IgD, IgE, IgG and IgM isotype antibodies, humanized antibodies, human antibodies, chimeric antibodies and antibody conjugates, and fragments of any of these.

Therapeutic enzymes include, inter alia, DNase, activated protein C, tissue plasminogen activator, and coagulation factors such as factor VIIa, factor IXa and factor Xa.

Proteins to which the present invention may be applied may contain naturally occurring L-amino acids or unnatural amino acids, such as D-amino acids. The amino acid sequence of the proteins may be identical to those occurring naturally in animals or other organisms or may be analogs in which the native sequence is altered in various ways. In analogs of proteins, one or more amino acids may be added, deleted or replaced by other amino acids at the N-terminal, C-terminal or internal portions of the protein. Analogs of proteins are well known in the art.

The term “precursor protein” refers to a protein which, using a combination of enzymatic, chemical or other reaction steps, may be transformed or converted into a therapeutic protein. Examples of precursor proteins include;

preproinsulin, which may be transformed into the therapeutic proteins proinsulin or insulin; proinsulin, which may be converted into the therapeutic protein insulin; and Met(B-1)Arg(B0)Lys(B28)Pro(B29)-human proinsulin, which may be transformed into the therapeutic protein Lys(B28)Pro(B29)-human insulin.

Proteins and precursor proteins to which the present invention may be applied may be generated by biosynthesis using recombinant DNA technology and are referred to herein as “recombinant proteins” or “recombinantly produced proteins”. The skilled reader will know how to use recombinant technology to biosynthesize the proteins and precursor proteins of the present invention.

Proteins and precursor proteins to which the present invention may be applied may also be prepared by chemical synthesis techniques, including classical solution phase methods, solid phase methods, semi-synthetic methods or other methods well known to those skilled in the art.

The term “folded protein” refers to a protein in its properly folded, three-dimensional conformation, and includes the designed, desired, or required arrangement of disulfide bonds linking the cysteine residues of the protein. Usually, this properly folded disulfide arrangement will be identical to or comparable to that present in its analogous native protein. For application of the present invention, the folded, target protein must not contain any cysteine residues in which the thiol moiety is not properly linked as a disulfide bond. Preferably, folded proteins stabilized by the process of the present invention will have two or more disulfide bonds.

The term “folded, recombinantly produced protein” refers to a folded protein produced by means of recombinant DNA technology. It comprises at least two cysteine residues linked together by a disulfide bond and contains no unlinked cysteine residues.

The term “contaminating protein” refers to a protein that has one or more cysteine residues in which the thiol moiety is unpaired. In a protein solution, contaminating proteins will typically be related in sequence to the desired folded protein but will differ by their disulfide bond arrangement and, in particular, by having at least one free thiol moiety. A contaminating protein may exist as a protein monomer, or as a dimer or polymer connected by disulfide bonds or other covalent linkages. A contaminating protein may also be present in an aggregated state in which the molecules are non-covalently bound together in a soluble or insoluble form.

The term “protein solution” is a liquid solution in which a quantity of protein is dissolved. A protein solution to which the process of the present invention may be applied comprises a folded protein and a contaminating protein.

The term “low molecular-weight thiol compound” refers to an organic compound that has a molecular weight of less than 1000 daltons and contains a single free thiol (SH) moiety. Examples of low molecular weight thiol compounds include, inter alia, cysteine, cysteamine, glutathione, 2-mercaptoethanol and 3-mercaptopropionic acid. For the present invention, a group of preferred low molecular weight thiol compounds consists of cysteine and 2-mercaptoethanol. A most preferred low molecular weight thiol compound is cysteine.

The term “total thiol moiety” refers to the sum of the thiol moieties in a protein solution present in the contaminating proteins and present in the low molecular weight thiol compounds within and/or added to the protein solution.

The term “oxidizing agent” refers to a chemical compound that gives up oxygen easily or removes hydrogen from another compound. Examples of oxidizing agents useful for the present invention include, inter alia, hydrogen peroxide, compounds containing the cupric ion such as cupric sulfate, cupric nitrate and cupric chloride, and compounds containing the ferric ion, such as ferric sulfate, ferric nitrate and ferric chloride. For the present invention, a group of preferred oxidizing agents consists of hydrogen peroxide and cupric sulfate. A most preferred oxidizing agent is hydrogen peroxide.

The term “effective amount” refers to a quantity of an oxidizing agent that increases the stability of a folded protein in a solution further comprising a contaminating protein and a low molecular weight thiol compound. Excessive quantities of an oxidizing agent should be avoided because such may cause chemical damage to the protein, especially oxidation at methionine, tryptophan, and histidine residues. An “optimal level” of an oxidizing agent is an effective amount of an oxidizing agent that achieves a near maximal increase in the stability of a protein solution.

An effective amount and an optimal level of an oxidizing agent to be added to stabilize a protein solution may be determined by methods such as those described below, by the procedures demonstrated in Examples 4 and 5, and by other methods known to those skilled in the art.

In one method of determining an effective amount or, preferably, an optimal level of an oxidizing agent, the quantity of total thiol moiety in the protein solution, which includes those thiol moieties present in the contaminating protein as well as those thiol moieties in the low molecular weight thiol compounds present in and/or added to the protein solution, is calculated or estimated from the total thiol content of the materials introduced into the solution. Alternatively, a thiol specific reagent, such as Ellman's reagent, 5,5′-dithio-bis-2-nitrobenzoic acid, may be used to analyze the protein solution to quantify the level of total, free thiol moieties in the solution.

From the total thiol moiety quantitation procedures noted above, determining an effective amount or, preferably, an optimal level, of an oxidizing agent to be added to a protein solution must take into account many factors, including the particular oxidizing agent. For hydrogen peroxide, which is consumed upon reaction with thiol moieties, one range of an effective amount to be added to a protein solution is about 0.2 to about 10 moles of H₂O₂ per mole of total thiol moiety present in the protein solution. A more preferred range of hydrogen peroxide is 0.35 to 2 moles per mole of total thiol moiety. A most preferred range of hydrogen peroxide is 0.4 to 0.55 moles per mole of total thiol moiety.

Oxidizing agents such as salts of the cupric and ferric ions are catalytic and, thus, are not consumed in reaction with thiol moieties. Therefore, low levels of oxidizing agents such as cupric sulfate and ferric chloride are generally more effective than comparable molar levels of hydrogen peroxide in stabilizing a protein solution.

A preferred method of determining an effective amount or, preferably, an optimal level of an oxidizing agent to be added to a protein solution is to utilize portions, or aliquots, of the protein solution to test different levels of an oxidizing agent. The test conditions used in this “empirical” approach should, preferably, mimic the storage conditions or processing steps that are planned for the remaining portion of the protein solution. The stability of the protein in the test aliquot, as well as formation of undesirable side products such as oxidation products formed from the starting protein, may be monitored by analytical techniques known to those of skill in the art. Especially sensitive sites of protein oxidation include the side chains of methionine, tryptophan, cysteine, and histidine residues. An optimal level of an oxidizing agent to be added to the protein solution is determined by selecting a quantity that maximizes recovery of the desired protein while minimizing side-product formation in the solution.

There are many parameters that influence the effective amount or optimal level of an oxidizing agent that should be added to stabilize a protein solution. Such parameters include temperature, time, solution composition, pH, level of agitation and the specific processing steps a protein solution will undergo after the stabilization procedure. For example, a protein solution that needs to be purified by reversed-phase HPLC chromatography at 30° C. at pH 8 in a process that lasts 2 days may be stabilized most efficiently by addition of an optimal level of an oxidizing agent, while a protein solution that needs to be stored at 4° C. for only 2 hours may be sufficiently stabilized by addition of a minimally effective amount of an oxidizing agent. Representative methods for determining effective amounts and optimal levels of oxidizing agents for use in the process of the present invention are described above and are further illustrated in Examples 4 and 5. Other methods known to those skilled in the protein, arts may also be applied.

A preferred group of proteins that will benefit from the present invention consists of native forms of insulin, proinsulin, leptin, growth hormone, and precursors and analogs thereof.

A more preferred group of proteins that will benefit from the present invention consists of proteins related to proinsulin and proinsulin analogs. This group of proteins includes human proinsulin, human preproinsulin, and other natural sequences of proinsulin proteins. This group of proteins also includes proinsulin analogs, which are proinsulin proteins wherein one or more amino acid replacements, deletions, insertions or extensions are made to a native proinsulin sequence. Examples of proinsulin analogs include, inter alia, Lys(B28)Pro(B29)-human proinsulin and Asp(B28)-human proinsulin.

Proinsulin analogs include precursor proteins such as Met(B-1)Arg(B0)Lys(B28)Pro(B29)-human proinsulin. In this protein nomenclature, the B-l and BO designate amino acids extended at the N-terminus of the proinsulin molecule, which is called the B-chain portion of the single chain protein. Lys(B28) and Pro(B29) designate single amino acid substitutions for the naturally occurring Pro(B28) and Lys(B29) amino acids in native human proinsulin.

Proinsulins, proinsulin analogs, and precursor proteins may be converted into intermediate forms and eventually into insulin and insulin analogs that are therapeutically useful in treating humans and other animals. Examples of such therapeutic proteins include human insulin, Lys(B28)Pro(B29)-human insulin, Asp(B28)-human insulin, Gly(A21)Arg(B31)Arg(B32)-human insulin and Lys(B3)Pro(B29)-human insulin. Such conversion reactions may include the use of hydrolytic enzymes, such as trypsin, chymotrypsin, carboxypeptidases, pepsin, and dipepidyl-diaminopeptidase (dDAP), as well as protein cleaving chemicals such as cyanogen bromide. Human insulin and insulin analogs themselves are also proteins that may benefit from the present invention if contaminating proteins are present at a level that causes stability problems in a solution comprising these proteins.

The insulin, proinsulin, proinsulin precursor, and related analog proteins described above have three disulfide bonds in their properly folded, or desired, conformation connecting the sulfur atoms of the following cysteine (Cys) residues: Cys(A6)-Cys(A11); Cys(A7)-Cys(B7); and Cys(A20)-Cys(B19). An example of a contaminating protein that may be present in a proinsulin protein solution would be one in which two disulfide bonds, Cys(A7)-Cys(B7) and Cys(A20)-Cys(B19), are intact, while the cysteine residues at A6 and All are present in free thiol form.

A protein solution that may benefit from the present invention is any solution comprising a folded protein and a contaminating protein as defined above. A suitable protein solution may contain protein material that has undergone a disulfide folding step and from which a portion of the contaminating protein has been removed, for example, by size exclusion chromatography. A suitable protein in solution may also be a precursor protein which requires, either before or after application of the process of the present invention, additional processing steps, such as enzymatic or chemical cleavage reactions, to convert the precursor protein into the proper protein structure useful as a therapeutic protein. A suitable protein solution may also comprise a purified, folded protein that, through various circumstances such as extended storage at alkaline pH, has become contaminated with a contaminating protein at a level that can cause stability problems.

A protein solution that may benefit significantly from the present invention is one derived from a disulfide folding reaction conducted on a protein produced from a bacterial source. A preferable bacterial source for the recombinant protein is a strain of E. coli that has been genetically modified to produce the desired protein. For illustrative purposes, see Frank, B. H., U.S. Pat. No. 4,430,266, issued Feb. 7, 1984.

The folding reaction itself may encompass a two-stage renaturation process involving a reduction step followed by a separate oxidation step, or a one-stage process wherein the reduction and oxidation steps are combined. Preferably, a starting protein solution that will benefit from the present invention is a completed folding reaction solution for which no additional purification steps have been conducted.

Preferably, a protein folding reaction, which is typically performed at neutral or alkaline pH, will comprise a low molecular weight thiol compound. Preferably, the completed folding reaction solution will comprise at least one mole of a low molecular weight thiol compound per mole of thiol moiety in the contaminating protein. A description of how size exclusion chromatography coupled with post-column derivatization may be used to quantify the thiol moiety content present in the contaminating protein and in the low molecular weight thiol compound is found in Examples 1 and 4. Other methods known to those skilled in the art may also be utilized. To the completed folding reaction solution is added an effective amount or, preferably, an optimal level of an oxidizing agent to stabilize the protein solution.

A protein solution that has undergone substantial purification to remove endogenous contaminating protein may also benefit from the present invention, if contaminating protein is still present at a level that can cause subsequent stability problems. Optionally, the present invention may be used to treat a previously purified protein solution which is subsequently found to contain a level of contaminating protein that leads to stability problems.

Preferably, a disulfide folding reaction solution that will benefit from the present invention will comprise a low molecular weight thiol compound such as cysteine, cysteamine, 2-mercaptoethanol, glutathione, or 3-mercaptopropionic acid. For each of these thiol compounds, the oxidizing agent will react with the free thiol groups of the contaminating protein and the low molecular weight thiol compound to form mixed disulfide adducts, as illustrated in FIG. 1. The low molecular weight thiol compound in the folding reaction solution is preferably cysteine or 2-mercaptoethanol. Preferably, the completed folding reaction solution will comprise at least one mole of low molecular weight thiol compound per mole of thiol moiety present in the contaminating protein.

If a starting protein solution to be treated by the process of the present invention does not contain a low molecular weight thiol compound, then a quantity of low molecular weight thiol compound, preferably cysteine or 2mercaptoethanol, is added to the protein solution, preferably before but, optionally, concomitantly with addition of the oxidizing agent, to promote reactions that lead to formation of mixed disulfide adducts. Preferably, at least one mole of low molecular weight thiol compound is added per mole of thiol moiety present in the contaminating protein. The thiol moiety present in the contaminating protein in a protein solution may be quantified using, for example, Ellman's reagent, 5,5′-dithio-bis-2-nitrobenzoic acid, or by using other thiol-specific reagents or techniques known to those skilled in the art. The exact timing of the addition of the low molecular weight thiol compound and the oxidizing agent is not critical, as long as they are able to react with each other and the free thiol groups of the endogenous contaminating protein to form mixed disulfide adducts.

A key requirement for a protein solution to be able to benefit from the process of the present invention is that, in addition to a properly folded protein, the solution must also comprise a protein contaminant in which one or more cysteine residues is present in free thiol form. The contaminating protein may represent from about 0.1% to about 90% of the total protein in the solution. The process of the invention has more applicability and value, however, when the contaminating protein represents about 5% to about 80% of the total protein. The process of the invention, in effect, lowers the level of contaminating protein in the solution by forming mixed disulfide adducts between the low molecular weight thiol compound and the free thiol groups of the contaminating protein.

In one embodiment of the present invention, a recombinant protein is produced in a bacteria such as E. coli. Subsequent to the fermentation stage, the protein, whose cysteine residues are preferably in an S-sulfonate form, undergoes disulfide bond formation in the presence of a low molecular weight thiol compound, which is preferably cysteine or 2-mercaptoethanol. Following completion of the folding reaction, which is preferably at about pH 7 to about pH 11.5, or more preferably at about pH 8 to about pH 11, an effective amount or, preferably, an optimal level of an oxidizing agent, preferably hydrogen peroxide, is added to the fold solution. The addition of the oxidizing agent increases the stability of the protein solution during subsequent storage and processing steps.

In another embodiment, a folded, recombinantly produced protein may undergo filtration, concentration, purification or enzyme or chemical cleavage steps, resulting in removal of most or all of the non-protein components present during the fold reaction. If a contaminating protein is still present at a level that can cause stability problems, the present invention may be employed to improve the stability of the protein solution. A sufficient quantity of a low molecular weight thiol compound is added to the solution prior to or concomitantly with addition of an effective amount or, preferably, an optimal level of an oxidizing agent. The free thiol groups of the contaminating protein form mixed disulfide adducts with the thiol groups of the low molecular weight thiol compound that, in effect, lowers the level of the free thiol-containing contaminating protein. The resulting protein solution is thus stabilized for subsequent storage or other protein processing steps.

Examples of protein processing steps are known to those skilled in the art, and include, inter alia; filtration through porous membranes or filters; concentration by diafiltration, ultrafiltration, or liquid evaporation; purification by reversed-phase HPLC chromatography, ion exchange chromatography, hydrophobic interaction chromatography, size exclusion chromatography or by crystallization; chemical cleavage using cyanogen bromide; and enzymatic cleavage using trypsin, chymotrypsin, carboxypeptidases, pepsin, subtilisin or dDAP. Storage in solution includes a wide range of solution components, temperatures and pH values.

During storage, protein solutions stabilized by the process of the present invention retain a greater quantity of the correctly folded protein compared to solutions to which the process of this invention was not applied. Furthermore, protein solutions stabilized by the process of the present invention provide a higher yield of the desired protein during subsequent filtration, concentration, purification, enzymatic cleavage or chemical cleavage procedures. Thus, under a variety of conditions and procedures, the invention provides a greater quantity of folded proteins, including therapeutic proteins useful for treating diseases in humans and other animals.

The present invention may be better understood with reference to the following examples. These examples are intended to be representative of specific embodiments of the invention and are not intended as limiting the scope of the invention.

### EXAMPLE 1

The Use of Hydrogen Peroxide to Stabilize a Concentrated Fold Reaction Solution at pH 8.2.

Folded, biosynthetic Met(B-1)Arg(B0)Lys(B28)Pro(B29)-human proinsulin (MR-KPB-HPI) was generated in a fold reaction by reacting its S-sulfonate form, MR-KPB-HPI-(SS03)6, at a concentration of 1 mg/mL, for about 18 to 20 hours at about 6° C. in an aqueous solution containing 2.1 mM cysteine at pH 10.9. Upon completion of the fold reaction, about 65% to about 70% of the total protein in solution was the correctly folded protein MR-KPB-HPI.

An acidified aliquot of the fold solution was analyzed by size exclusion chromatography on a Superose-12 HR 10/30 (Amersham Pharmacia Biotech, Inc., Piscataway, N.J., USA) column using 7 M urea, 20 mM phosphoric acid, 50 mM NaCl buffer at pH 3.2 for elution. Post-column derivatization with Ellman's reagent, 5,5′-dithio-bis-2-nitrobenzoic acid, at alkaline pH and subsequent monitoring at 421 nm were employed to quantify peaks containing free thiol moieties. This analysis showed the presence of a large cysteine peak and also showed that most of the contaminating protein (i.e., protein possessing one or more free thiol groups) eluted as MR-KPB-HPI-related dimers and polymers.

To an untreated portion of the completed fold solution was added an aliquot of a 3% (0.88 M) hydrogen peroxide solution to obtain a final hydrogen peroxide concentration of 1.2 mM. Storage of this solution proceeded for 30 minutes at about 6° C. A control solution was provided by leaving a portion of the completed fold reaction untreated. Each of the test solutions was then acidified to pH 3 and concentrated by ultrafiltration using an Amicon S1Y3 filter (Millipore Corp., Bedford, Mass., USA) to a protein concentration of about 10 mg/mL. Each concentrate was adjusted to pH 8.2 and warmed to ambient temperature (about 20° C. to about 25° C.). At various times, aliquots of the solution were removed and diluted 25-fold with 0.1 M glycine pH 3 buffer. The percent of MR-KPB-HPI remaining at each time point, compared to the amount present prior to adjustment to pH 8.2, was determined by analyzing the acidified solutions using reversed-phase HPLC chromatography on a 4.6 mm×150 mm Zorbax C-8 column (Mac-Mod Analytical, Inc., Chadds Ford, Pa., USA). Protein was eluted from the column with an acetonitrile gradient in a buffer solution consisting of 35.6 mM octane sulfonic acid sodium salt, 52.2 mM phosphoric acid and 0.0723% morpholine at about pH 2, and the eluant was monitored at 214 nm. The results of this experiment are depicted in Table 1.

TABLE 1
Percent of MR-KPB-HPI remaining in test solutions
at pH 8.2 with or without H₂O₂ treatment.

Percent MR-KPB-HPI Remaining

| Storage Time | Untreated | Hydrogen Peroxide |
|---|---|---|
| (hours) | Sample | Treated Sample |
| 0.25 | 71 | 97 |
| 0.5 | 55 | 94 |
| 1 | 33 | 91 |
| 2 | 18 | 90 |
| 3.7 | 10 | 89 |
| 5.3 | 4 | 85 |
| 6.7 | 3 | 84 |

These data clearly show that the addition of hydrogen peroxide stabilized the protein solution under the pH 8.2, 20° C. to 25° C. test conditions. In the untreated sample, loss of MR-KPB-HPI predominantly resulted from protein polymerization induced by the thiol moiety of the endogenous contaminating protein.

### EXAMPLE 2

Evidence of Cysteine-Protein Disulfide Adducts Formed After Treatment of KPB-HPI and Cysteine with Hydrogen Peroxide.

A sample of purified, biosynthetic Lys(B28)Pro(B29)-human proinsulin (KPB-HPI) at 9.6 mg/mL in 7 M urea at pH 10.9 was reduced with 20 mM cysteine for 20 minutes at 6° C. A solution of hydrogen peroxide was then added to the protein solution to obtain a final H₂O₂ concentration of 10 mM. After about-20 minutes at ambient temperature (about 20° C. to about 25° C.) the reaction solution was acidified to about pH 3.

A portion of the solution was then chromatographed by reversed-phase HPLC chromatography using a 4.6 mm×150 mm Zorbax C-8 column (Mac-Mod Analytical, Inc., Chadds Ford, Pa., USA). The protein was eluted with an acetonitrile gradient in 0.1% TFA buffer. The eluant was monitored by mass spectrometry using an LCQ model ion trap mass spectrometer (Finnigan Corporation, San Jose, Calif., USA). The mass spectral analysis of the protein peak is shown in FIG. 1.

The chromatogram in FIG. 1 indicates the presence of mixed disulfide adducts in which the KPB-HPI protein is covalently linked to 2, 4 or 6 cysteine moieties as well as a dimer of the protein covalently linked to 2 cysteine moieties.

### EXAMPLE 3

Evaluation of Cupric Sulfate as an Oxidizing Agent.

Biosynthetic Met(B-1)Arg(B0)Lys(B28)Pro(B29)-human proinsulin (MR-KPB-HPI) was generated in a fold reaction as described in Example 1. A portion of the completed fold solution was adjusted to pH 8.2. Then, various quantities of cupric sulfate solutions were added to aliquots of the pH 8.2 solution to obtain final cupric sulfate concentrations of 0.01 mM, 0.1 mM and 1 mM. A control sample was provided by leaving a portion of the completed fold reaction untreated. After incubating the test samples for 1.9 hours at 4° C. to 8° C., the test solutions were diluted 3-fold with a 0.1 M glycine pH 3 solution.

The percent of MR-KPB-HPI remaining in the test solutions was determined by analyzing the acidified test solutions by reversed-phase HPLC chromatography as described in Example 1. The level of free thiol remaining in the protein components in the test solutions was determined by size exclusion chromatography on a Superose-12 HR 10/30 column as described in Example 1. The data from these test solutions are shown in Table 2. The untreated control sample described above was also analyzed to determine the starting levels of the protein-related thiol and MR-KPB-HPI.

TABLE 2
Percent, relative to untreated control, of
MR-KPB-HPI and protein-related thiol remaining in
a fold solution containing various levels of Cu₂SO₄.

| Cupric Sulfate | Protein-Related | |
|---|---|---|
| Concentration | Thiol | MR-KPB-HPI |
| 0.01 mM | 75 | 107 |
| 0.1 mM | 12 | 100 |
| 1 mM | 8 | 85 |

This experiment showed that a relatively low quantity of cupric sulfate, 0.1 mM, significantly lowered the level of thiol-containing contaminating protein in the MR-KPB-HPI solution. The 1 mM level of cupric sulfate, however, led to some loss of MR-KPB-HPI protein due to oxidation of the methionine and possibly other residues in the protein.

### EXAMPLE 4

Optimization of Hydrogen Peroxide Levels to Stabilize Folded Protein Solutions at Various pH Values.

A folding solution of MR-KPB-HPI was prepared as described in Example 1. Portions of the completed fold reaction were adjusted to pH 3, pH 8 and a portion remained at pH 10.9. To aliquots of these solutions were added various quantities of a 3% hydrogen peroxide solution to final concentrations of up to 10 mM H₂O₂. A control aliquot was left untreated to determine the starting MR-KPB-HPI and protein-related thiol levels.

After storage for 1.3 hours at 6° C., the oxidation reactions were stopped by adding to each solution an equal volume of a 0.1 M methionine, 500 mM phosphoric acid solution, resulting in solutions of about pH 2.5 to about pH 3.0. The protein-related thiol content remaining in each sample was determined by analytical size exclusion chromatography on a G25 Sephadex Superfine column (Amersham Pharmacia Biotech Inc., Piscataway, N.J., USA) using the same eluting buffer and monitoring procedures as described for the Superose-12 HR 10/30 column in Example 1. The percent of the protein product, MR-KPB-HPI, remaining in each test sample was determined by reversed-phase HPLC chromatography as described in Example 1. The results of this experiment are shown in Table 3.

TABLE 3
Percent of protein (MR-KPB-HPI) and protein-related
thiol remaining in fold solutions adjusted to various
pH values and containing various levels of H₂O₂.

| | | | |
|---|---|---|---|
| H₂O₂ | pH 3 | pH 8 | pH 10.9 |

| (mM) | Thiol | Protein | Thiol | Protein | Thiol | Protein |
|---|---|---|---|---|---|---|
| 0 | 100 | 100 | 83 | 100 | 94 | 100 |
| 0.1 | 93 | 99 | 66 | 100 | 73 | 103 |
| 0.3 | 86 | 99 | 43 | 99 | 43 | 102 |
| 1 | 51 | 98 | 12 | 97 | 13 | 100 |
| 2 | 48 | 96 | 10 | 94 | 7 | 99 |
| 3 | 46 | 95 | 9 | 93 | 9 | 97 |
| 10 | 47 | 85 | 8 | 81 | 5 | 87 |

These data clearly show that addition of hydrogen peroxide to the folded protein solution lowered the protein-related thiol content of the samples stored at all three pH values. The solutions treated and held at alkaline pH values had a more complete formation of mixed disulfide adducts involving the protein-related thiol groups (indicated by loss of protein-related thiol) than the acidic solution. At all pH values, hydrogen peroxide at the 1 mM to 3 mM level achieved a near maximal lowering of the thiol content of the contaminating protein and, in addition, near maximal levels of the MR-KPB-HPI protein remained.

These data also showed that the quantity of MR-KPB-HPI protein in solution decreased if an excessive, that is, 10 mM, level of hydrogen peroxide was present. This was chiefly due to increased oxidation of the methionine residue in the protein.

Therefore, in viewing the data in Table 3, this experiment empirically determined that an effective amount of hydrogen peroxide that may be added to lower the thiol content of the contaminating protein and preserve most of the desired MR-KPB-HPI protein, over a wide range of pH values, is about 0.3 mM to about 10 mM. The experiment also determined that, under the conditions examined, an optimal level of hydrogen peroxide that maximizes removal of the contaminating protein and preserves most of the MR-KPB-HPI is about 1 mM to about 3 mM.

### EXAMPLE 5

Optimization of Hydrogen Peroxide Level in Stabilizing a Folded Protein Solution.

A folding solution of MR-KPB-HPI was prepared as described in Example 1. To aliquots of the completed fold solution were added various quantities of a 3% hydrogen peroxide solution to final concentrations of 0.6 to 1.5 mM. After storage for 30 minutes at pH 10.9 at 6° C., the solutions were adjusted to about pH 3 with HCl and concentrated about 8-fold using Amicon Centriprep 3,000 MW cut-off centrifugal concentrators (Millipore Corp., Bedford, Mass., USA). A control sample for this experiment was an aliquot of the fold solution which was not treated with hydrogen peroxide, but was subsequently acidified to about pH 3 and concentrated as described for the test samples.

The concentrated test samples were evaluated to determine the protein-related thiol content remaining, compared to the untreated control sample, by analytical size exclusion chromatography on a G25 Sephadex Superfine column as described in Example 4.

Portions of the concentrated test samples were also stored for 140 hours at pH 3 at 6° C. These aliquots were then analyzed for the increased content, as a percent of total protein, of M(O)R-KPB-HPI by the reversed-phase HPLC chromatography system described in Example 1. M(O)R-KPB-HPI is MR-KPB-HPI in which the methionine residue is oxidized.

Portions of the concentrated test samples were also adjusted to about pH 8 and stored at ambient temperature (20° C. to 25° C.) for 4.4 hours. The samples were then diluted 25-fold with a 0.1 M glycine pH 3 solution. The acidified samples were analyzed for the percent of MR-KPB-HPI remaining compared to a pre-pH adjusted control sample.

The results of these analyses are shown in Table 4.

TABLE 4
Percent of MR-KPB-HPI and protein-related thiol
remaining and percent increase in M(O)R-KPB-HPI content
in stored solutions containing various levels of H₂O₂.

| | Remaining | Increase in | Remaining |
|---|---|---|---|
| | MR-KPB-HPI | M(O)R-KPB-HPI | Protein-Related |
| H₂O₂ | (4.4 hr, pH 8, | Content | Thiol (0.5 hr, |
| (mM) | 20° C. to 25° C.) | (140 hours, pH 3, 6° C.) | pH 10.9, 6° C.) |
| 0 | 51.0 | 0 | 100 |
| 0.6 | 78.0 | 0.4 | 22.6 |
| 0.7 | 82.0 | 0.5 | 18.7 |
| 0.8 | 84.0 | 0.6 | 13.4 |
| 0.9 | 87.0 | 0.3 | 10.3 |
| 1.0 | 87.5 | 0 | 7.5 |
| 1.1 | 90.0 | 0.1 | 5.2 |
| 1.2 | 88.0 | 3.8 | 2.7 |
| 1.3 | 89.0 | 6.1 | 2.8 |
| 1.5 | 89.0 | 11.8 | 2.8 |

The data in Table 4 show results that are useful in empirically determining the effective amount and optimal level of hydrogen peroxide to add to the completed protein fold solution to maximize its stability and minimize degradation due to oxidation.

The data in column 2 show the protein solution stabilized with hydrogen peroxide levels of 0.9 mM to 1.5 mM retained 87% to 90% of the desired protein, MR-KPB-HPI, while less of the desired protein was retained at lower H₂O₂ levels. The data in column 3 show that only minimal quantities of M(O)R-KPB-HPI formed at hydrogen peroxide levels up to 1.1 mM. The data in column 4 show that about 10% or less of the free thiol groups in the contaminating protein in the protein solutions remained as free thiols in the solutions having hydrogen peroxide levels of 0.9 mM to 1.5 mm.

In selecting an optimal level of hydrogen peroxide to add to similar protein solutions, the data from column 2 (remaining MR-KPB-HPI) and column 3 (formation of M(O)R-KPB-HPI) are most important. Combining results from these two parameters clearly points to an optimal level of hydrogen peroxide of 0.9 mM to 1.1 mM, a range that, under the conditions examined, maximizes recovery of the desired protein, MR-KPB-HPI, and minimizes formation of the oxidized methionine by-product. This experiment also demonstrates that an effective amount of hydrogen peroxide that provides some protection to the MR-KPB-HPI protein in the solutions, under the conditions examined, is 0.6 mM to 1.5 mM.

### EXAMPLE 6

Improved Protein Recovery in Process Steps Following Stabilization of a Fold Solution with Hydrogen Peroxide.

Biosynthetic Met(B-1)Arg(B0)Lys(B28)Pro(B29)-human proinsulin (MR-KPB-HPI) was generated in a fold reaction as described in Example 1.

To one portion of the completed fold solution was added an aliquot of a 3% hydrogen peroxide solution to obtain a final hydrogen peroxide concentration of 1 mM. A portion of the fold reaction solution which was not treated with hydrogen peroxide served as a control solution. After 30 minutes at pH 10.9 at 6° C., the test and control solutions were acidified to pH 3 with HCl and the yield of MR-KPB-HPI was determined by reversed-phase HPLC chromatography as described in Example 1.

The acidified solutions were then concentrated by ultrafiltration using Amicon Spiral Wound filters (Millipore Corp., Bedford, Mass., USA). Upon completion of this step, the step recovery yield of MR-KPB-HPI in the test and control samples was quantified by reversed-phase HPLC chromatography as described in Example 1.

Dipepidyl-diaminopeptidase, or DDAP, from Dictyostelium discoideum, described by Atkinson, P. R., et al., in U.S. Pat. No. 5,565,349, issued Oct. 15, 1996, was then added to the acidic protein solutions to effect the removal of the Met-Arg (MR) dipeptide from the N-terminus of the protein in a procedure described by-Atkinson, P. R., et al., in U.S. Pat. No. 5,565,330, issued Oct. 15, 1996. The step recovery yield of KPB-HPI was quantified by reversed-phase HPLC chromatography as described in Example 1.

The average yields and standard deviations from four separate runs of the processing steps are shown in Table 5.

TABLE 5
Percent step-wise yields for three successive process
steps conducted on a completed fold solution of the
protein MR-KPB-HPI, with and without H₂O₂ treatment.

| | |
|---|---|
| Control | H₂O₂ Treated |

| Process Step | Yield | SD | Yield | SD |
|---|---|---|---|---|
| Storage of Completed | 66.8 | 1.7 | 68.5 | 2.2 |
| Fold Solution | | | | |
| Concentration of | 89.5 | 6.4 | 90.1 | 8.5 |
| Acidified Solution | | | | |
| dDAP Removal | 93.9 | 2.5 | 99.0 | 3.2 |
| of Met-Arg | | | | |

SD = Standard Deviation

These data show that the addition of hydrogen peroxide to the completed fold solution improved the yield of protein after each of the subsequent processing steps. The average step-wise yields of the desired proteins were higher in the hydrogen peroxide treated samples, as determined by HPLC analysis, following further storage of the completed fold solution (product is MR-KPB-HPI), through the acidified solution concentration step (product is MR-KPB-HPI) and through the removal of the Met-Arg dipeptide using the dDAP enzyme (product is KPB-HPI).

I claim:

1. A process for increasing the stability of a protein solution comprising a folded protein and a contaminating protein, comprising adding to the protein solution a low molecular weight thiol compound and an effective amount of an oxidizing agent.

2. A process for increasing the stability of a protein solution comprising a folded protein, a contaminating protein, and a low molecular weight thiol compound, comprising adding to the protein solution an effective amount of an oxidizing agent.

3. The process according to any one of claims 1 or 2, wherein the folded protein is a folded, recombinantly produced protein.

4. The process according to any one of claims 1 to 3, wherein the effective amount of the oxidizing agent is determined empirically.

5. The process according to any one of claims 1 to 4, wherein the effective amount of the oxidizing agent is an optimal level of the oxidizing agent.

6. The process according to any one of claims 1 to 5, wherein the low molecular weight thiol compound is selected from the group consisting of cysteine, cysteamine, glutathione, 2-mercaptoethanol and 3-mercaptopropionic acid.

7. The process according to claim 6, wherein the low molecular weight thiol compound is cysteine.

8. The process according to any one of claims 1 to 7, wherein the protein solution is at neutral or alkaline pH.

9. The process according to any one of claims 1 to 8, wherein the oxidizing agent is selected from the group consisting of hydrogen peroxide, cupric sulfate, cupric nitrate, cupric chloride, ferric sulfate, ferric nitrate and ferric chloride.

10. The process according to claim 9, wherein the oxidizing agent is selected from the group consisting of hydrogen peroxide and cupric sulfate.

11. The process according to claim 10, wherein the oxidizing agent is hydrogen peroxide.

12. The process according to claim 11, wherein 0.2 to 10 moles of hydrogen peroxide is added per mole of total thiol moiety.

13. The process according to claim 12, wherein 0.35 to 2 moles of hydrogen peroxide is added per mole of total thiol moiety.

14. The process according to claim 13, wherein 0.4 to 0.55 moles of hydrogen peroxide is added per mole of total thiol moiety.

15. The process according to any one of claims 1 to 14, wherein the quantity of the low molecular weight thiol compound present in or added to the protein solution is at least one mole per mole of thiol moiety in the contaminating protein.

16. The process according to any one of claims 1 to 15, wherein the folded protein is a hormone, an antibody or an enzyme, or a precursor or analog thereof.

17. The process according to claim 16, wherein the folded protein is a hormone, or a precursor or an analog thereof.

18. The process according to claim 17, wherein the folded protein is a native form of insulin, proinsulin, leptin, growth hormone, or a precursor or analog thereof.

19. The process according to claim 18, wherein the folded protein is human proinsulin, a precursor of human proinsulin, a proinsulin analog or a precursor of a proinsulin analog.

20. The process according to claim 19, wherein the folded protein is a precursor of human proinsulin or a precursor of a proinsulin analog.

21. The process according to claim 20, wherein the folded protein is Met(B-1)Arg(B0)Lys(B28)Pro(B29)-human proinsulin.

22. The process according to claim 20, wherein the folded protein is a precursor of human proinsulin and further comprising one or more subsequent steps wherein the precursor of human proinsulin is converted to human insulin.

23. The process according to claim 20, wherein the folded protein is a precursor of a proinsulin analog and further comprising one or more subsequent steps wherein the precursor of a proinsulin analog is converted to an insulin analog.

24. The process according to claim 21, further comprising one or more subsequent steps wherein the Met(B-1)Arg(B0)Lys(B28)Pro(B29)-human proinsulin is converted to Lys(B28)Pro(B29)-human insulin.

25. A process for making human insulin or an insulin analog, comprising:
a) adding a low molecular weight thiol compound and an effective amount of an oxidizing agent to a protein solution comprising a folded precursor of human insulin or a folded precursor of an insulin analog, and a contaminating protein; and
b) converting the folded precursor protein to human insulin or an insulin analog.

26. A process for making human insulin or an insulin analog, comprising:
a) adding an effective amount of an oxidizing agent to a protein solution comprising a folded precursor of human insulin or a folded precursor of an insulin analog, a contaminating protein and a low molecular weight thiol compound; and
b) converting the folded precursor protein to human insulin or an insulin analog.
